(19) United States
(12) Patent Application Publication
(10) Pub. No.: US 2007/0248486 A1
(43) Pub. Date: Oct. 25, 2007

(54) Process for Sterilization of Cyanoacrylate Adhesive Compositions and Devices

(75) Inventor: Carlos Morales, Cumming, GA (US)

Correspondence Address: ALTIMATIA, L.L.C., 16 ELM RIDGE ROAD, PENNINGTON, NJ, 08534, US

(73) Assignee: CLAST TRADING LTD, Hong Kong (CN)

(21) Appl. No.: 11/379,994

(22) Filed: Apr. 24, 2006

(51) Int. Cl. A61K 31/785; A61L 2/00

(52) U.S. Cl. 422/22; 424/78.27

(57) ABSTRACT

The present invention provides a novel method of sterilizing 2-Cyanoacrylate ester compositions using a combination of chemical and irradiation means, and the resulting novel compositions. The combination of monomeric 2-Cyanoacrylate and irradiation have a lethal effect on microbials, rendering sterilized compositions when the appropriate sterilization condition is achieved and when the method is applied to 2-Cyanoacrylates in sealed containers. Samples are sterilized using electron beam at doses as low as about 1 to about 5 Kgy.

As embodied and described herein, the present invention provides a novel method of sterilizing 2-Cyanoacrylate ester compositions using a combination of chemical and irradiation means, and the resulting novel compositions. The combination of monomeric 2-Cyanoacrylate and irradiation have a lethal effect on microbials, rendering sterilized compositions when the appropriate sterilization condition is achieved and when the method is applied to 2-Cyanoacrylates in sealed containers.

As used herein, the following terms have the following meanings. The term “cyanoacrylate adhesive composition” or “cyanoacrylate adhesive compositions” refers to polymerizable formulations comprising polymerizable cyanoacrylate ester monomers. The term aldose is intended to refer to both common disacharides and monosacharides.

In one embodiment of the invention, 2-cyanoacrylate adhesive compositions are sterilized through an unexpected and heretofore unknown combination of irradiation dose, sterilizing at e-beam doses significantly lower than previously thought to be effective. Previous e-beam sterilization methods have required doses of over 15 to 20 kGy. Under the present invention, the 2-cyanoacrylate adhesive compositions can be sterilized at doses of from about 1 to 15 kGy. As would be expected, the dose selected to accomplish the sterilization will depend on product density and electron beam time exposure.

A minimum dose of about 1 to about 5 kGy is necessary to accomplish sterilization. Published US patent application 2006/0062687, which is herein incorporated by reference, describes methods for assaying cyanoacrylate compositions for the effectiveness of dry heat sterilization of cyanoacrylate compositions for dry heat sterilization processes at temperatures of from about 70° C. to about 140° C.

Samples containing formulated n-Butyl and 2-Octyl cyanoacrylate with and without thickener (poly-cyanoacrylate) in sealed borosilicate glass, and high density polyethylene, were inoculated prior to sterilization with commercially available Bacillus subtillis contained in cotton threads (Biological Indicators or BI's) at a concentration of 1×10⁶. Among the commercially available biological indicators which may be used are: bacterial spores on a stainless steel disc, bacterial spores on a steel wire, bacterial spores on steel coupons, bacterial spores on borosilicate paper and bacterial spores on woven cotton threads. Among the species of spores which may be chosen for use in the commercially available biological indicators are Bacillus subtillis and Geobacillus Stearothermophillus. Commercially available biological indicators may be obtained from any commercial supplier, such as Raven Labs. Some inoculated glass vials and tubes samples were kept at room temperature without sterilization as positive controls, while the rest of the samples were sterilized at doses of from about 1 to about 5, about 5 to about 10, and about 10 to about 15 kGy. Samples were sent to a microbiology laboratory for determination of the presence or absence of growth after the sterilization procedure was completed to assay the effectiveness of the process conditions.

In assaying one embodiment of the invention microorganisms are utilized which may be killed by the sterilization process but which show significant resistance to this process. The term microorganism refers to bacteria, fungi, yeast, protozoa algae, viruses and protozoa. Bacterial spores are very resistant to heat and chemicals; more so than vegetative bacterial cells, therefore the spores are often used to monitor sterilization procedures. In one embodiment of the invention, an appropriate organism for monitoring e-beam sterilization is Bacillus subtillis.

The spores represent a resting stage in the life cycle of the Bacillus genus. The resting spore contains a large number of active enzymes which allow the transformation from dormant cell to vegetative cell. The germination process, or the return to the vegetative state, has been described as a time-ordered sequence involving activation, triggering, initiation and outgrowth. Activation is reversible and involves an increase in the rate and extent of germination. Triggering is irreversible and is the result of spore contact with the germinant. Initiation involves the loss of heat resistance, release of dipicolinic acid and calcium, loss of refractility and absorbance. Outgrowth results in formation of the vegetative cell.

In accordance with the present invention a cyanoacrylate composition test sample comprising at least one sterility test strip, or lyophilized spores is utilized. While reference is made to “spores” as a test microorganism it should be understood that microorganisms other than spore formers may be used in conjunction with the present invention. The spore strips utilized with the present invention are preferably constructed of a material which is inert to the microorganisms and inert to cyanoacrylate monomer. A variety of commercial spore strips are readily available and can be utilized with the present invention. The spore strips can contain more than one type of microorganism.

To assay the sterilized samples and controls, the compositions including the biological indicators are transferred into containers filled with an aqueous aldose solution, shaken, and transferred into a quantity of nutrient medium in an aseptic container. Transferring the samples to an aldose solution serves to emulsify the cyanoacrylate monomer without causing it to polymerize as it would upon exposure to water alone. Aldoses which act to emulsify the cyanoacrylate include without limitation, dextrose, lactose, arabinose, mannose, galactose, rhamnose, fructose, sucrose and glucose. In one embodiment of the invention, the aldose is dextrose. The concentration of the aldose solution may be from about 2% to about 50% on a weight/weight basis. A preferred range for the concentration of the aldose solution is from about 3% to about 15%. A more preferred aldose concentration is from about 5% to about 10% weight/weight. The nutrient medium supports the germination of spores and growth of any viable microorganisms. The nutrient medium contains a protein substrate for the proteases liberated during spore germination and during subsequent microbial growth. The nutrient medium preferably comprises an aqueous solution or suspension of nutrient components (including the protein substrate) needed in order to promote the growth of viable microorganisms that may exist after the sterilization process. One example of a suitable culture medium is a protein containing microbiological broth such as tryptic soy broth (TSB) and/or TSB with specific protein additives, such as, for example casein. Formulations for culture media are well-known to those in the art.

The mixture of microorganisms, cyanoacrylate, aldose and nutrient medium are then sealed within a containing means. The samples are then incubated for a predetermined period of time at from about 28° C. to about 37° C. Any microorganisms not killed during the sterilization process begin to germinate and grow during the incubation period. In a preferred embodiment the microorganisms are incubated for at least about seven days. Thereafter the sample is examined to detect the presence of growth by different methods, such as visual examination of the samples followed by microscope Gram stain examination, addition of an enzymatic indicator such as Tetrazolium salts followed by UV spectrophotometric analysis, or direct UV spectrophotometric analysis of incubated samples. In one embodiment, after visual examination a gram stain smear is prepared to look for gram positive rods which would confirm growth. In another embodiment, growth can be determined by the addition of enzymatic biological indicator such as Tetrazolium salts, wherein bacterial activity is determined by development of color which may be measured quantitatively with a Ultraviolet spectrophotometer at 257 nm. In yet another embodiment, a sample without enzymatic indicator is analyzed under a spectrophotometer at a wavelength of 480 nm to determine growth.

The method of the invention can be applied in principle to any 2-cyanoacrylate ester monomer. In one embodiment of the invention, the 2-cyanoacrylate is an aliphatic cyanoacrylate ester and preferably an alkyl, cycloalkyl, alkenyl, alkoxyalkyl, fluroroalkyl, fluorocyclic alkyl or fluoroalkoxy 2-cyanoacrylate ester. The alkyl group may contain from 2 to 12 carbon atoms, and is preferably a C₂ to C₈ alkyl ester, and is most preferably a C₄ to C₈ alkyl ester. Suitable 2-cyanoacrylate esters include without limitation, the ethyl, n-propyl, iso-propyl, n-butyl, pentyl, hexyl, cyclohexyl, heptyl, n-octyl, 2-ethylhexyl, 2-methoxyethyl and 2-ethoxyethyl esters. Any of these 2-cyanoacrylate monomers may be used alone, or they may be used in mixtures.

The 2-cyanoacrylate monomers of the invention can be prepared by any of the methods known in the art. U.S. Pat. Nos. 2,721,858, 3,254,111 and 4,364,876 each of which is hereby incorporated in its entirety by reference, disclose methods for preparing 2-cyanoacrylates. For example, cyanoacrylates for the instant invention were prepared by reacting cyanoacetate with formaldehyde in the presence of heat and a basic condensation catalyst to give a low molecular weight polymer. A depolymerization step followed under heat and vacuum in the presence of acidic and anionic inhibitors, yielding a crude monomer that could be distilled under vacuum and in the presence of radical and acidic inhibitors. The distilled 2-cyanoacrylate monomers are then formulated with radical and acidic inhibitors depending upon their application and to provide the necessary stability.

The 2-cyanoacrylate compositions of the invention may in some embodiments contain a thickening agent to increase the viscosity of the composition. This thickening agent may be a polymer. The thickening agent may be selected from the group consisting of without limitation, poly alkyl-2-cyanoacrylates, poly cycloalkyl-2-cyanoacrylates, poly fluoroalkyl-2-cyanoacrylates, poly fluorocycloalkyl-2-cyanoacrylates, poly alkoxyalkyl-2-cyanoacrylates, poly alkoxycycloalkyl-2-cyanoacrylates, poly fluoroalkoxyalkyl-2-cyanoacrylates, polyalkoxycyclofluoroalkyl-2-cyanoacrylates, poly vinyl acetate, poly lactic acid and poly gylcolic acid. In order to obtain optimum solubility of the polymer in the monomer, the polymer is often chosen to be a polymer of the monomer or one of the monomers which comprise the 2-cyanoacrylate composition. Preferably, the polymer is soluble in the monomer composition at ambient temperature. Preferred polymers include polymers of octyl 2-cyanoacrylate, methyl methacrylate, vinyl acetate lactic acid, or glycolic acid. The preferred weight average molecular weight of the polymers is from about 100,000 to about 2,000,000. More preferably, the polymer molecular weight is from about 200,000 to about 1,600,000.

Cyanoacrylate polymers of the invention can be prepared by slow addition of the monomer to a mixer containing 0.1% Sodium Hydroxide or Sodium Bicarbonate in deionized water. Water is then decanted away, and the polymer is rinsed several times with deionized water and decanted again. Following steps include neutralizing the polymer with 0.1N HCL, rinsing with deionized water, drying on a vacuum heated oven at temperature of less than 80° C. and grinding polymer to fine particles.

The amount of thickening agent that is added to the monomer composition is dependent upon the molecular weight of the polymer, and the desired viscosity for the adhesive composition. The thickening agent typically is added at from about 1% to about 25% by weight of the composition. Preferably it is added at from about 1% to about 10%. More preferably it is added at from about 1% to about 5%. A typical viscosity of the composition is from about 25 to about 3000 centipoise, as measured by a Brookfield viscometer at 25° C. Preferably, the viscosity is between from about 50 to about 600 centipoise at 25° C. The specific amount of a given thickening agent to be added can be determined by one of ordinary skill in the art without undue experimentation.

The 2-Cyanoacrylate compositions may contain one or more acidic inhibitors in the range from 1 to 1,000 ppm. Such acidic inhibitors include without limitation: sulfur dioxide, nitrogen oxide, boron Oxide, phosphoric acid, ortho, meta, or para-phosphoric acid, acetic acid, benzoic acid, cyanoacetic acid, tri-fluoroacetic acid, tribromoacetic acid, trichloroacetic acid, boron trifluoride, hydrogen fluoride, perchloric acid, hydrochloric acid, hydrobromic acid, sulfonic acid, fluorosulfonic acid, chlorosulfonic acid, sulfuric acid, and toluenesulfonic acid.

The 2-Cyanoacrylate compositions may contain one or more free radical polymerization inhibitors in the range from 0 to 10,000 ppm. Examples of such radical inhibitors include without limitation, catechol; hydroquinone; hydroquinone monomethyl ether and hindered phenols such as Butylated Hydroxyanisol; Butylated hydroxytoluene (2,6-di-tert-butyl butylphenol and 4-methoxyphenol); 4-ethoxyphenol; 3 methoxyphenol; 2-tert-butyl-4methoxyphenol; 2,2methylene-bis-(4-methyl-6-tert-butylphenol).

The 2-Cyanoacrylate compositions may contain single or mixtures of plasticizers such as tributyl acetyl citrate; Tributyl citrate; dimethyl sebacate; diethyl sebacate; try-ethyl phosphate; tri-(2-ethylhexyl)phosphate; tri-cresyl phosphate; glyceryl triacetate; glyceryl tributyrate; dioctyl adipate; isopropyl myristate; butyl stearate; trioctyl trimellitate and dioctyl glutarate. The plasticizers may be added to the compositions in proportions of less than 50% w/w of the formulation.

The 2-Cyanoacrylate compositions may contain small amounts of dyes like the derivatives of anthracene and other complex structures. Some of these dyes include without limitation, 1-hydroxy-4-[4-methylphenylamino]-9,10 anthracenedione (D&C violet No. 2); disodium salt of 6-hydroxy-5-[(4-sulfophenyl)axo]-2-naphthalene-sulfonic acid (FD&C Yellow No.6,); 9-(o-carboxyphenyl)-6-hydroxy-2,4,5,7-tetraiodo-3H-xanthen-3-one,disodium salt, monohydrate (FD&C Red No.3); 2-(1,3dihydro-3-oxo-5-sulfo-2H-indole-2-ylidine)-2,3-dihydro-3-oxo-1H-indole-5-sulfonic acid disodium salt (FD&C Blue No.2); and [phthalocyaninato (2)]copper. add in proportions of less than 50000 ppm.

The sterilized cyanoacrylate adhesive compositions of the invention may be packaged in a container made of any suitable material. Suitable materials must be irradiation stable and resistant up to the sterilization optimal dose, must provide an adequate barrier to atmospheric moisture and be compatible with the cyanoacrylate monomer or monomers. Materials meeting these requirements include metals and borosilicate type I glass. Suitable metals can include without limitation aluminum, tin and stainless steel. Metals can have different forms like pouches and tubes. Glass can be used as vials, breakable tubes or any other shape, and contained inside tubes made out of the same material, or combinations or materials including plastics. Particularly preferred materials are aluminum, type I glass and plastics. Preferred aluminum tubes comprise a nozzle which is hermetically sealed by a pierceable membrane of aluminum and are filled at their end remote from the nozzle prior to closure of the open end by tight crimping. The glass vials used in this invention, are made out of borosilicate type I glass and sealed with a threaded phenolic cap with a silicone/teflon septa or sealed with an aluminum crimp cap and silicon/teflon septa. Also, the plastic bottles used in this invention are made of high density polyethylene with polypropylene nozzles and high density polyethylene. In the result, therefore, preferred embodiments of the invention reside in a substantially hermetically sealed aluminum container, e.g. An aluminum tube, containing a sterile 2-cyanoacrylate composition, a or type I glass vials hermetically sealed with a phenolic threaded and silicone/teflon and a plastic bottle/cap containers made of high density polyethylene (HDPE) with a polypropylene nozzle.

### EXAMPLES

Sample Testing: (Sterility Test Method for all Samples)

The method was tested by first performing the USP bacteriostasis and fungistasis test on glass vials and aluminum tubes and plastic bottles. The sterility test was performed by obtaining cotton threads biological indicators (BI's) with a population of Bacillus subtillis var niger of 1×10⁶ ml. Spores. The biological indicators were placed in glass vial, and plastic bottles. These cyanoacrylate containers with the biological indicators were filled with 0.5 to 0.6 ml of the corresponding cyanoacrylate necessary for the sterilization trials. Some of these containers were not sterilized and designated as controls. The rest of the containers were exposed to different radiation doses. Non sterilize and sterilized cyanoacrylate samples plus BI's were transferred to a 5% Dextrose USP solution, shaken and transferred to Soy Casein Digested Broth (SCDB) and incubated at 35-37° C. for at least seven days.

Polymer Preparation: (Polymer Method for Samples Containing Polymer)

2-OCA polymer was made by adding drop by drop 30 grams of 2-OCA monomer to a blender containing 1000 ml of 0.1% Sodium bicarbonate deionized water while swirling. Bicarbonate water with the polymer was vacuum filtered on a Kitasato with a Fisherbrand #Q8 quantitative filter paper, rinsed five times with 500 ml aliquots of deionize water, decanted and polymer neutralized with 500 ml of 0.1 N Hydrochloric acid. The neutralized polymer was rinsed with three aliquots of 500 ml, decanted, dried in a vacuum oven at 80° C. and fine grinded with mixer after drying.

Sample Composition Preparation:

### Example I

A sample mixture of 2-Octyl/n-Butyl cyanoacrylate (60/40% rate show) containing 2-Octyl cyanoacrylate polymer was made by mixing 2-Octyl/n-Butyl cyanoacrylates (stabilized with 100 ppm of SO₂, 3000 ppm of Butylated Hydroxyanisole) with 3.5% of 2-OCA polymer. The polymer was dissolved in the formulated cyanoacrylate by heating and mixing in a round glass flask equipped with a paddle shaft and mixer at a temperature no higher than 80° C. and obtaining a viscosity of 340 cP (measured with Brookfield DV-II at 25° C). Then, the composition was inoculated with Bacillus subtillis cotton threads BI's with a population of 1×10⁶ which were filled in glass type I glass threaded vials and plastic HDPE bottles. vials and plastic bottles were filled by hand with an Eppendorf automatic pipette. The vials, were sealed with threaded phenol caps/silicone/Teflon septa (glass vials) and the plastic bottles with HDPE threaded cap. Some cyanoacrylate inoculated glass and plastic samples were not sterilized and used as positive Standard Biological Indicators to indicate livable spores. The rest of the cyanoacrylate inoculated and sealed vials and plastics bottles were exposed to the irradiation doses stipulated in the sterilization testing protocol condition.

Groups of class vials were placed inside 110 mm×100 mm polyethylene plastic bags. Plastic HDPE bottles containing cyanoacrylate adhesive were placed inside 110 mm×100 mm polyethylene plastic bags. All plastic bags with their corresponding vials or bottles were placed flat on a conveyor to achieve one layer exposure to an electron beam accelerator set up at a power of 10 MeV. The plastic bags with the samples and dosimeter radiachromic film were placed on the conveyor and the samples were irradiated at from about 1 to 5, about 5 to 10, about 10 to 15 or about 15 to 20 Kgy. After irradiation, the films were read in a spectrophotometer and the irradiation absorption was measure in nanometers and then translated into dose in kilograys. Dosimeter film readings were 4.4-4.6, 7.0-7.4, 9.2-9.7, 13.6-14.3, and 16.8-17.7 Kgy. After sterilization, viscosity was measured on a Brookfield DVII rheometer at 25° C. and the results noted in the tables for the specific examples. Setting times were measured on sheep plasma at 25° C. with polymerization occurring at less than one second. Gas chromatography was performed with normal results of more than 99 percent purity and BHA content also within specifications.

Tables #1-2-show example results.

TABLE #1
60/40 2-OCA/n-BCA + polymer sterilization example packed
in glass vials with pre-sterilization viscosity of 340 cP

| | | Type of | Incubation | Number | Number of | | Viscosity @ |
|---|---|---|---|---|---|---|---|
| Dose | Type of | media | temperature | samples | days | Number of | 25° C. |
| kGy | sample | 400 ml | ° C. | tested | incubated | positives | sterile |
| No dose | Control 60/40 | SCDB | 30-35 | 3 | 2 | 3 | |
| | (2-OCA/n-BCA + | | | | | | |
| | polymer) + BI | | | | | | |
| 1-5 | 60/40 (2-OCA/n-BCA + | SCDB | 30-35 | 3 | 7 | 0 | 336 |
| | polymer) + BI | | | | | | |
| 5-10 | 60/40 2-OCA/n-BCA + | SCDB | 30-35 | 3 | 7 | 0 | 320 |
| | polymer) + BI | | | | | | |
| 10-15 | 60/40 2-OCA/n-BCA + | SCDB | 30-35 | 3 | 9 | 0 | 297 |
| | polymer) + BI | | | | | | |
| 15-20 | 60/40 (2-OCA/n-BCA + | SCDB | 30-35 | 3 | 9 | 0 | 286 |
| | polymer) + BI | | | | | | |

Table #1 above shows minimum sterilization doseages and the results obtained for glass vials samples of 60/40% 2OCA/n/BCA with 2-OCA polymer and cotton threads BI's with Bacillus subtillis spores

TABLE #2
60/40 2-OCA/n-BCA sterilization example packed in plastic
bottles with pre-sterilization viscosity of 340 cP.

| | | Type of | Incubation | Number | Number of | | Viscosity @ |
|---|---|---|---|---|---|---|---|
| Dose | Type of | media | temperature | samples | days | Number of | 25° C. |
| kGy | sample | 400 ml | ° C. | tested | incubated | positives | sterile |
| No dose | Control 60/40 | SCDB | 30-35 | 3 | 2 | 3 | |
| | (2-OCA/n-BCA + | | | | | | |
| | polymer) + BI | | | | | | |
| 1-5 | 60/40 (2-OCA/n-BCA + | SCDB | 30-35 | 3 | 7 | 0 | 344 |
| | polymer) + BI | | | | | | |
| 5-10 | 60/40 (2-OCA/n-BCA + | SCDB | 30-35 | 3 | 7 | 0 | 326 |
| | polymer) + BI | | | | | | |
| 10-15 | 60/40 (2-OCA/n-BCA + | SCDB | 30-35 | 3 | 9 | 0 | 287 |
| | polymer) + BI | | | | | | |
| 15-20 | 60/40 (2-OCA/n-BCA + | SCDB | 30-35 | 3 | 9 | 0 | 283 |
| | polymer) + BI | | | | | | |

Table #2 above shows minimum sterilization doseages and the results obtained for plastic samples of 60/40% 2OCA/n/BCA with 2-OCA polymer and cotton threads BI's with Bacillus subtillis spores

### Example II

Sample Composition Preparation

A sample mixture of n-Butyl cyanoacrylate (n-BCA) was made by stabilizing with 100 ppm of SO₂ and 3000 ppm of Butylated Hydroxyanisole. Then, the composition was inoculated with Bacillus subtillis cotton threads BI's with a population of 1×10⁶ which were filled in glass type I threaded vials and plastic HDPE bottles. Vials and plastic bottles were filled by hand with an Eppendorf automatic pipette. The vials were sealed with threaded phenol caps/silicone/Teflon septa (glass vials) and the plastic bottles with HDPE threaded cap. Some cyanoacrylate inoculated glass and plastic samples were not sterilized and used as positive Standard Biological Indicators to indicate livable spores. The rest of the cyanoacrylate inoculated and sealed vials and plastics bottles were exposed to the irradiation doses stipulated in the sterilization testing protocol condition.

Tables #3-4 show example results.

TABLE #3
n-BCA sterilization example packed in glass vials
with pre-sterilization viscosity of 2.76 cP

| | | Type of | Incubation | Number | Number of | | Viscosity @ |
|---|---|---|---|---|---|---|---|
| Dose | Type of | media | temperature | samples | days | Number of | 25° C. |
| kGy | sample | 400 ml | ° C. | tested | incubated | positives | sterile |
| No dose | Control | SCDB | 30-35 | 3 | 2 | 3 | |
| | n-BCA + BI | | | | | | |
| 1-5 | n-BCA | SCDB | 30-35 | 3 | 7 | 0 | 2.63 |
| 5-10 | n-BCA | SCDB | 30-35 | 3 | 7 | 0 | 2.86 |
| 10-15 | n-BCA | SCDB | 30-35 | 3 | 9 | 0 | 2.83 |
| 15-20 | n-BCA | SCDB | 30-35 | 3 | 9 | 0 | 2.91 |

Table #3 above shows minimum sterilization doseages and the results obtained for glass vials samples of n/BCA and cotton threads BI's with Bacillus subtillis spores

TABLE #4
n-BCA sterilization example packed in plastic bottles
with pre-sterilization viscosity of 2.76 cP

| | | Type of | Incubation | Number | Number of | | Viscosity @ |
|---|---|---|---|---|---|---|---|
| Dose | Type of | media | temperature | samples | days | Number of | 25° C. |
| kGy | sample | 400 ml | ° C. | tested | incubated | positives | sterile |
| No dose | Control | SCDB | 30-35 | 3 | 2 | 3 | |
| | n-BCA + BI | | | | | | |
| 1-5 | n-BCA + BI | SCDB | 30-35 | 3 | 7 | 0 | 2.76 |
| 5-10 | n-BCA + BI | SCDB | 30-35 | 3 | 7 | 0 | 2.81 |
| 10-15 | n-BCA + BI | SCDB | 30-35 | 3 | 7 | 0 | 2.78 |
| 15-20 | n-BCA + BI | SCDB | 30-35 | 3 | 9 | 0 | 2.89 |

Table #4 above shows minimum sterilization doseages and the results obtained for plastic samples of n/BCA and cotton threads BI's with Bacillus subtillis spores

### Example III

Sample Composition Preparation

A sample of 2-Octyl cyanoacrylate (2-OCA) was made by stabilizing with 100 ppm of SO₂ and 3000 ppm of Butylated Hydroxyanisole. Then, the composition was inoculated with Bacillus subtillis cotton threads BI's with a population of 1×10⁶ which were filled in glass type I threaded vials and plastic HDPE bottles. Vials and plastic bottles were filled by hand with an Eppendorf automatic pipette. The vials were sealed with threaded phenol caps/silicone/Teflon septa (glass vials) and the plastic bottles with HDPE threaded cap. Some cyanoacrylate inoculated glass and plastic samples were not sterilized and used as positive Standard Biological Indicators to indicate livable spores. The rest of the cyanoacrylate inoculated and sealed vials and plastics bottles were exposed to the irradiation doses stipulated in the sterilization testing protocol condition.

Tables #-5-6 show example results.

TABLE #5
2-OCA sterilization example packed in glass vials
with pre-sterilization viscosity of 5.82 cP

| | | Type of | Incubation | Number | Number of | | Viscosity @ |
|---|---|---|---|---|---|---|---|
| Dose | Type of | media | temperature | samples | days | Number of | 25° C. |
| kGy | sample | 400 ml | ° C. | tested | incubated | positives | sterile |
| No dose | Control | SCDB | 30-35 | 3 | 2 | 3 | |
| | 2-OCA + BI | | | | | | |
| 1-5 | 2-OCA + BI | SCDB | 30-35 | 3 | 7 | 0 | 5.81 |
| 5-10 | 2-OCA + BI | SCDB | 30-35 | 3 | 7 | 0 | 6.1 |
| 10-15 | 2-OCA + BI | SCDB | 30-35 | 3 | 7 | 0 | 6.5 |
| 15-20 | 2-OCA + BI | SCDB | 30-35 | 3 | 9 | 0 | 6.4 |

Table #5 above shows minimum sterilization doseages and the results obtained for glass vials samples of 2-OCA and cotton threads BI's with Bacillus subtillis spores

TABLE #6
2-OCA sterilization example packed in plastic with pre-sterilization viscosity of 5.82 cP

| | | Type of | Incubation | Number | Number of | | Viscosity @ |
|---|---|---|---|---|---|---|---|
| Dose | Type of | media | temperature | samples | days | Number of | 25° C. |
| kGy | sample | 400 ml | ° C. | tested | incubated | positives | sterile |
| No dose | Control | SCDB | 30-35 | 3 | 2 | 3 | |
| | 2-OCA + BI | | | | | | |
| 1-5 | 2-OCA + BI | SCDB | 30-35 | 3 | 7 | 0 | 5.85 |
| 5-10 | 2-OCA + BI | SCDB | 30-35 | 3 | 7 | 0 | 6.8 |
| 10-15 | 2-OCA + BI | SCDB | 30-35 | 3 | 9 | 0 | 6.7 |
| 15-20 | 2-OCA + BI | SCDB | 30-35 | 1 | 9 | 0 | 7.2 |

Table #6 above shows minimum sterilization doseages and the results obtained for plastic samples of 2-OCA and cotton threads BI's with Bacillus subtillis spores

### Example IV

To test the performance of the Bacillus subtillis cotton threads BI's with a population of 1×10⁶ spores, the BI's were placed and sealed with no cyanoacrylate (CA) inside empty glass type I threaded vials with treaded caps and on plastic HDPE bottles. Some of the glass vials and HDPE plastic bottles were not irradiated but tested for livable spores suspended on cotton treads and another group of glass vials and HDPE plastic bottles with the BI's were exposed to an e-beam irradiation of 1-5, 5-10,10-15 and 15-20 kGy with the objective of testing the spores survivability to e-beam irradiation. Tables #7-8 show example results.

TABLE #7
Non irradiated/irradiated BI's no CA
controls performance in glass vials

| | | | | | | Number |
|---|---|---|---|---|---|---|
| | | Type of | Incubation | Number | Number of | of |
| Dose | Type of | media | temperature | samples | days | posi- |
| kGy | sample | 400 ml | ° C. | tested | incubated | tives |
| None | Non | SCDB | 30-35 | 3 | 2 | 3 |
| | irradiated | | | | | |
| | control | | | | | |
| 1-5 | Irradiated | SCDB | 30-35 | 3 | 3 | 3 |
| | control | | | | | |
| 5-10 | Irradiated | SCDB | 30-35 | 3 | 9 | 0 |
| | control | | | | | |
| 10-15 | Irradiated | SCDB | 30-35 | 3 | 9 | 0 |
| | control | | | | | |
| 15-20 | Irradiated | SCDB | 30-35 | 3 | 9 | 0 |
| | control | | | | | |

TABLE #8
Non irradiated/irradiated BI's no CA
controls performance in plastic vials

| | | | | | | Number |
|---|---|---|---|---|---|---|
| | | Type of | Incubation | Number | Number of | of |
| Dose | Type of | media | temperature | samples | days | posi- |
| kGy | sample | 400 ml | ° C. | tested | incubated | tives |
| None | Non | SCDB | 30-35 | 3 | 2 | 3 |
| | irradiated | | | | | |
| | control | | | | | |
| 1-5 | Irradiated | SCDB | 30-35 | 3 | 3 | 3 |
| | control | | | | | |
| 5-10 | Irradiated | SCDB | 30-35 | 3 | 9 | 0 |
| | control | | | | | |
| 10-15 | Irradiated | SCDB | 30-35 | 3 | 9 | 0 |
| | control | | | | | |
| 15-20 | Irradiated | SCDB | 30-35 | 3 | 9 | 0 |
| | control | | | | | |

What is claimed is:

1. A method of sterilization of 2-cyanoacrylate adhesive compositions, comprising;
irradiation of the composition in a device at a e-beam dose of about 1 to about 10 kGy sufficient to sterilize the composition.

2. A method as in claim 1 wherein the e-beam dose is from about 1 to about 5 kGy.

3. A method according to claim 1 wherein said cyanoacrylate composition comprises one or more 2-cyanoacrylate ester monomers wherein said cyanoacrylate ester monomer is an alkyl, cycloalkyl, fluoroalkyl, fluorocycloalkyl or fluoroalkoxy 2-cyanoacrylate monomer.

4. A method according to claim 1 wherein said cyanoacrylate composition further comprises at least one thickener selected from the group consisting of poly alkyl-2-cyanoacrylates, poly cycloalkyl-2-cyanoacrylates, poly fluoroalkyl-2-cyanoacrylates, poly fluorocycloalkyl-2-cyanoacrylates, poly alkoxyalkyl-2-cyanoacrylates, poly alkoxycycloalkyl-2-cyanoacrylates, poly fluoroalkoxyalkyl-2-cyanoacrylates, polyalkoxycyclofluoroalkyl-2-cyanoacrylates, poly lactic acid and poly gylcolic acid.

5. A method according to claim 4 wherein the at least one thickener is a poly alkyl-2-cyanoacrylate.

6. A method according to claim 5 wherein the alkyl group of the poly alkyl-2-cyanoacrylate is selected from the group consisting of straight chain or branched chain C₄ to C₈ hydrocarbons.

7. A method according to claim 5 wherein said thickening polymer is poly octyl 2-cyanoacrylate.

8. A method according to claim 2 wherein the 2-cyanoacrylate composition further comprises a stabilizer.

9. A method according to claim 8 wherein said stabilizer is one or more of an anionic polymerization inhibitor or a free radical polymerization inhibitor.

10. A method according to claim 4 wherein said cyanoacrylate composition further comprises a plasticizer selected from the group consisting of tributyl acetyl citrate, dimethyl sebacate, diethyl sebacate, triethyl phosphate, tri-(2-ethylhexyl)phosphate, tricresyl phosphate, glycerol triacetate, glycerol tributyrate, dioctyl adipate, isopropyl myristate, butyl stearate, trioctyl trimellitate and dioctyl glutarate.

11. A method according to claim 10 wherein said plasticizer is up to 50% by weight of the cyanoacrylate composition.

12. A sterilized cyanoacrylate adhesive composition comprising:
a 2-cyanoacrylate ester monomer;
wherein the cyanoacrylate adhesive composition is sterilized by irradiation of the composition in a device at a e-beam dose of about 1 to about 10 kGY sufficient to sterilize the composition.

13. A composition as in claim 12 wherein the e-beam dose is from about 1 to about 5 kGy.

14. A composition as in claim 12 wherein said cyanoacrylate composition comprises one or more 2-cyanoacrylate ester monomers wherein said cyanoacrylate ester monomer is an alkyl, cycloalkyl, fluoroalkyl, fluorocycloalkyl or fluoroalkoxy 2-cyanoacrylate monomer.

15. A composition according to claim 12 further comprising at least one thickener selected from the group consisting of poly alkyl-2-cyanoacrylates, poly cycloalkyl-2-cyanoacrylates, poly fluoroalkyl-2-cyanoacrylates, poly fluorocycloalkyl-2-cyanoacrylates, poly alkoxyalkyl-2-cyanoacrylates, poly alkoxycycloalkyl-2-cyanoacrylates, poly fluoroalkoxyalkyl-2-cyanoacrylates, polyalkoxycyclofluoroalkyl-2-cyanoacrylates, poly lactic acid and poly gylcolic acid.

16. A composition according to claim 15 wherein the thickener is a poly alkyl-2-cyanoacrylate.

17. A composition according to claim 16 wherein the alkyl group of the poly alkyl-2-cyanoacrylate is selected from the group consisting of straight chain or branched chain C₄ to C₈ hydrocarbons.

18. A composition according to claim 16 wherein the thickener is poly octyl-2-cyanoacrylate.

19. A composition according to claim 12 wherein the 2-cyanoacrylate composition further comprises a stabilizer.

20. A composition according to claim 19 wherein said stabilizer is one or more of an anionic polymerization inhibitor or a free radical polymerization inhibitor.

21. A composition according to claim 12 wherein said cyanoacrylate composition further comprises a plasticizer selected from the group consisting of tributyl acetyl citrate, Tributyl citrate, dimethyl sebacate, diethyl sebacate, triethyl phosphate, tri-(2-ethylhexyl)phosphate, tricresyl phosphate, glycerol triacetate, glycerol tributyrate, dioctyl adipate, isopropyl myristate, butyl stearate, trioctyl trimellitate and dioctyl glutarate.

22. A composition according to claim 21 wherein said plasticizer is up to 50% by weight of the cyanoacrylate composition.
